(19) United States
(12) Patent Application Publication
(10) Pub. No.: US 2005/0075629 A1
(43) Pub. Date: Apr. 7, 2005

(54) Apparatus and method for assessing tissue ablation transmurality

(75) Inventors: Pierre-Antoine Chapelon, Fremont, CA (US); Dany Berube, Milpitas, CA (US)

Correspondence Address: FENWICK & WEST LLP, SILICON VALLEY CENTER, 801 CALIFORNIA STREET, MOUNTAIN VIEW, CA, 94041, US

(73) Assignee: AFX, INC., FREMONT, CA (US)

(21) Appl. No.: 10/370,179

(22) Filed: Feb. 19, 2003

Related U.S. Application Data
(60) Provisional application No. 60/358,215, filed on Feb. 19, 2002.

(51) Int. Cl. A61B 18/18

(52) U.S. Cl. 606/41

(57) ABSTRACT

An instrument is provided to assess the transmurality of an ablation lesion from a first surface of a targeted biological tissue to an opposed second surface thereof. The instrument includes a needle member having an elongated shaft and a distal tip portion adapted to pierce the tissue first surface and into the ablation lesion of the biological tissue. A plurality of needle electrodes are spaced-apart along the elongated shaft. When the needle member pierces the tissue first surface, each the electrode being positioned at different respective depths of the biological tissue from the tissue first surface to the tissue second surface. These electrodes each measure at least one of conduction time, conduction velocity, phase angle, and impedance through at least a portion of the targeted tissue and at the respective depth to determine the transmurality of the ablation lesion.

### RELATED APPLICATION DATA

The present application claims priority under 35 U.S.C. §119 to U.S. Provisional Application Ser. No. 60/358,215, naming Chapelon et al. inventors, and filed Feb. 19, 2002, and entitled TRANSMURALITY ASSESSMENT DEVICE, the entirety of which is incorporated herein by reference in its entirety for all purposes.

### BACKGROUND OF THE INVENTION

1. Field of Invention

The present invention relates, generally, to tissue ablation instruments and lesion formation thereof, and more particularly, relates to apparatus and methodology for assessing tissue ablation transmurality.

2. Description of the Prior Art

It is well documented that atrial fibrillation, either alone or as a consequence of other cardiac disease, continues to persist as the most common cardiac arrhythmia. According to recent estimates, more than two million people in the U.S. suffer from this common arrhythmia, roughly 0.15% to 1.0% of the population. Moreover, the prevalence of this cardiac disease increases with age, affecting nearly 8% to 17% of those over 60 years of age.

Atrial arrhythmia may be treated using several methods. Pharmacological treatment of atrial fibrillation, for example, is initially the preferred approach, first to maintain normal sinus rhythm, or secondly to decrease the ventricular response rate. Other forms of treatment include drug therapies, electrical cardioversion, and RF catheter ablation of selected areas determined by mapping. In the more recent past, other surgical procedures have been developed for atrial fibrillation, including left atrial isolation, transvenous catheter or cryosurgical ablation of His bundle, and the Corridor procedure, which have effectively eliminated irregular ventricular rhythm. However, these procedures have for the most part failed to restore normal cardiac hemodynamics, or alleviate the patient's vulnerability to thromboembolism because the atria are allowed to continue to fibrillate. Accordingly, a more effective surgical treatment was required to cure medically refractory atrial fibrillation of the Heart.

On the basis of electrophysiologic mapping of the atria and identification of macroreentrant circuits, a surgical approach was developed which effectively creates an electrical maze in the atrium (i.e., the MAZE procedure) and precludes the ability of the atria to fibrillate. Briefly, in the procedure commonly referred to as the MAZE III procedure, strategic atrial incisions are performed to prevent atrial reentry circuits and allow sinus impulses to activate the entire atrial myocardium, thereby preserving atrial transport function postoperatively. Since atrial fibrillation is characterized by the presence of multiple macroreentrant circuits that are fleeting in nature and can occur anywhere in the atria, it is prudent to interrupt all of the potential pathways for atrial macroreentrant circuits. These circuits, incidentally, have been identified by intraoperative mapping both experimentally and clinically in patients.

Generally, this procedure includes the excision of both atrial appendages, and the electrical isolation of the pulmonary veins. Further, strategically placed atrial incisions not only interrupt the conduction routes of the common reentrant circuits, but they also direct the sinus impulse from the sinoatrial node to the atrioventricular node along a specified route. In essence, the entire atrial myocardium, with the exception of the atrial appendages and the pulmonary veins, is electrically activated by providing for multiple blind alleys off the main conduction route between the sinoatrial node to the atrioventricular node. Atrial transport function is thus preserved postoperatively as generally set forth in the series of articles: Cox, Schuessler, Boineau, Canavan, Cain, Lindsay, Stone, Smith, Corr, Change, and D'Agostino, Jr., The Surgical Treatment Atrial Fibrillation (pts. 1-4), 101 THORAC CARDIOVASC SURG., 402-426, 569-592 (1991).

While this MAZE III procedure has proven effective in treating medically refractory atrial fibrillation and associated detrimental sequelae, this operational procedure is traumatic to the patient since this is an open-heart procedure and substantial incisions are introduced into the interior chambers of the Heart. Consequently, other techniques have been developed to interrupt atrial fibrillation restore sinus rhythm. One such technique is strategic ablation of the atrial tissues and lesion formation through tissue ablation instruments.

Most approved tissue ablation systems now utilize radio frequency (RF) energy as the ablating energy source. Accordingly, a variety of RF based catheters and power supplies are currently available to electrophysiologists. However, radio frequency energy has several limitations including the rapid dissipation of energy in surface tissues resulting in shallow “burns” and failure to access deeper arrhythmic tissues. Another limitation of RF ablation catheters is the risk of clot formation on the energy emitting electrodes. Such clots have an associated danger of causing potentially lethal strokes in the event that a clot is dislodged from the catheter. It is also very difficult to create continuous long lesions with RF ablation instruments.

As such, instruments which utilize other energy sources as the ablation energy source, for example in the microwave frequency range, are currently being developed. Microwave frequency energy, for example, has long been recognized as an effective energy source for heating biological tissues and has seen use in such hyperthermia applications as cancer treatment and preheating of blood prior to infusions. Accordingly, in view of the drawbacks of the traditional catheter ablation techniques, there has recently been a great deal of interest in using microwave energy as an ablation energy source. The advantage of microwave energy is that it is much easier to control and safer than direct current applications and it is capable of generating substantially larger and longer lesions than RF catheters, which greatly simplifies the actual ablation procedures. Such microwave ablation systems are described in the U.S. Pat. No. 4,641,649 to Walinsky; U.S. Pat. No. 5,246,438 to Langberg; U.S. Pat. No. 5,405,346 to Grundy, et al.; and U.S. Pat. No. 5,314,466 to Stem, et al, each of which is incorporated herein by reference.

Regardless of the energy source applied to ablate the arrhythmic tissues, these strategically placed lesions must electrically sever the targeted conduction paths. Thus, not only must the lesion be properly placed and sufficiently long, it must also be sufficiently deep to prevent the electrical impulses from traversing the lesion. Ablation lesions of insufficient depth may enable currents to pass over or under the lesion, and thus be incapable of disrupting the reentry circuits. In most cases, accordingly, it is desirable for the ablation lesion to be transmural.

To effectively disrupt electrical conduction through the cardiac tissue and gap junctions, which are regions of low electrical resistance, the tissue temperature must reach a threshold where irreversible cellular damage occurs. The temperature at the margin between viable and nonviable tissue has been demonstrated to be about 48° C. to about 5020 C. Haines D E, Watson D D, Tissue Heating During Radiofrequency Catheter Ablation: A Thermodynamic Model and Observations in Isolated Perfused and Superfused Canine Right Ventricular Free Wall, PACINT CLIN ELECTROPHYSIOL, June 1989, 12(6), pp. 962-76.)

Thus, to ensure ablation, the tissue temperature should exceed this margin. This, however, is often difficult to perform and/or assess since the cardiac tissue thickness varies with location and, further, varies from one individual to another.

Most tissue ablation instruments typically ablate tissue through the application of thermal energy directed toward a targeted biological tissue, in most cases the surface of the biological tissue. As the targeted surface of the biological tissue heats, for example, the ablation lesion propagates from the targeted surface toward an opposed second surface of the tissue. Excessive thermal energy at the interface between the tissue and the ablation head, on the other hand, is detrimental as well. For example, particularly with RF energy applications, temperatures above about 100° C. can cause coagulation at the RF tip. Moreover, the tissue may adhere to the tip, resulting in tearing at the ablation site upon removal of the ablation instrument, or immediate or subsequent perforation may occur. Thin walled tissues are particularly susceptible.

Generally, if the parameters of the ablation instrument and energy output are held constant, the lesion size and depth should be directly proportional to the interface temperature and the time of ablation. However, the lag in thermal conduction of the tissue is a function of the tissue composition, the tissue depth and the temperature differential. Since these variables may change constantly during the ablation procedure, and without overheating the tissues at the interface, it is often difficult to estimate the interface temperature and time of ablation to effect a proper transmural ablation, especially with deeper arrhythmic tissues.

Several attempts have been made to assess the completion or transmurality of an ablation lesion. The effective disruption of the electrical conduction of the tissue does of course affect the electrical characteristics of the biological tissue. Thus, some devices and techniques have been developed which attempt to measure at least one of the electrical properties, such as those based upon a function of impedance (e.g., its value, the change in value, or the rate of change in value) of the ablated tissue, to determine whether the ablation is transmural and complete. Typical of these devices include U.S. Pat. No. 6,322,558 to Taylor et al. and U.S. Pat. No. 5,403,312 to Yates et al.; U.S. patent application Ser. No. 09/747,609 to Hooven; and WIPO Pub. No. WO 01/58373 A1 to Foley et al., each of which is incorporated by reference in its entirety.

While these recent applications have been successful in part, they all tend to measure the electrical properties of the targeted ablation tissue directly from the surfaces of the tissue (i.e., the top surface or the underside surface of the tissue). This may be problematic since the measurement of such electrical properties can produce false indications with respect to transmurality of the ablation; a decrease in the change of impedance measured across the lesion indicative of transmurality, however, knowing there is insufficient energy applied to truly created a transmural lesion, as one example.

Accordingly, it would be advantageous to provide an apparatus and method to better assess the transmurality of an ablation lesion during an ablation procedure, for instance, by measuring the electrical properties along the depth of the lesion itself.

### SUMMARY OF THE INVENTION

The present invention provides a surgical device or instrument useful for facilitating tissue ablation procedures of sensitive biological tissue such as those of internal organs. In particular, the present invention is suitable for assessing the transmurality of an ablation lesion formed from a first surface of cardiac tissue of the heart to an opposed second surface thereof to electrically isolate conduction paths thereof during treatment of arrhythmia.

The instrument includes a needle member having an elongated shaft and a distal tip portion adapted to pierce the tissue first surface. A plurality of needle sensors are spaced-apart along the elongated shaft so that when the needle member is advanced into the targeted tissue from the tissue first surface toward the tissue second surface, each of the needle sensors is positioned at different respective depths of the biological tissue, and can selectively transmit or receive electrical signals. These measurements can then be analyzed to determine the transmurality or effectiveness of the ablation procedure.

Accordingly, by collectively analyzing this measured data, a surgeon may gauge whether an ablation procedure has been properly performed. Unlike the current transmurality assessment procedures, the present invention is capable of conducting measurements at varying depths of the targeted tissue so that very detailed analysis can be conducted.

In one specific embodiment, the needle sensors are provided by needle electrodes applied to measure the electrical characteristics of the local tissue to measure at least one of conduction time, conduction velocity, phase angle, and impedance through at least a portion of the targeted tissue at the respective depth. Using this information, audio or visual feedback may be provided to determine the ablation transmurality, or other lesion characteristic. In other examples, the feedback information may be applied for automatic closed-loop control of a tissue ablation instrument.

The present invention may further include a first side electrode and a second side electrode to engage the tissue first surface at spaced-apart locations. Further, these locations radially spaced from a longitudinal axis of the shaft to measure the at least one of conduction time, conduction velocity, phase angle, and impedance with respect to one or more needle electrodes.

In another embodiment, the first side electrode is supported at the distal end of a first support member extending radially away from the longitudinal axis of the shaft, while the second side electrode is supported at the distal end of a second support member also extending radially away from the longitudinal axis of the shaft. Each side electrode is supported in a manner such that when the shaft of the needle member is driven into the ablation lesion to position each needle electrode at the different respective depths of the biological tissue, at least one of the first side electrode and the second side electrode engage the tissue first surface.

The proximal portions of the first support member and the second support member are mounted to the shaft about 180° apart from one another along the longitudinal axis of the shaft. Further, the first side electrode and the second side electrode are sufficiently radially spaced from the longitudinal axis of the shaft so that the first side electrode can engage the first tissue surface on one side of the ablation lesion, and the second side electrode can engage the first tissue surface on an opposite other side of the ablation lesion.

In another aspect of the present invention, a tissue ablation assembly is provided that is adapted to ablate a targeted biological tissue from a first surface thereof to an opposed second surface thereof to form an ablation lesion. The ablation assembly includes an elongated transmission line having a proximal portion suitable for connection to an energy source. An antenna assembly is coupled to the transmission line, and is adapted to transmit energy therefrom sufficiently strong to cause tissue ablation at the first surface. A manipulating device may be included which cooperates with the ablation assembly for manipulative movement thereof. A needle member is further included having an elongated shaft and a distal tip portion adapted to pierce the tissue first surface and be advanced into the biological tissue. A plurality of needle electrodes are spaced apart along the elongated shaft such that when the needle member pierces the tissue first surface, each needle electrode is positioned at a different respective depth of the biological tissue from the tissue first surface to the tissue second surface. These needle electrodes are utilized to selectively transmit and/or receive electrical signals to measure at least one of conduction time, conduction velocity, phase angle, and impedance through at least a portion of the targeted tissue, at the respective depth, to determine the transmurality of the ablation lesion created or being created therein.

In yet another aspect of the present invention, a method is provided for assessing the transmurality of an ablation lesion from a first surface of a targeted biological tissue to an opposed second surface thereof. The method includes piercing a needle member having an elongated shaft into the targeted tissue from the tissue first surface. The needle member includes a plurality of needle electrodes spaced apart along the elongated shaft which are capable of transmitting or receiving electrical signals. When the needle member pierces into the ablation lesion, the electrodes are placed at different respective depths of the biological tissue from the tissue first surface to the tissue second surface. The method further includes selectively transmitting and/or receiving electrical signals from one or more needle electrodes to measure at least one of conduction time, conduction velocity, phase angle, and impedance through at least a portion of the targeted tissue, at the respective depth, to determine the transmurality of the ablation lesion created or being created. By analyzing this measured data, the degree of tissue ablation may be determined. Further, the piercing and/or transmitting or receiving may be performed before, during or after the creation of the ablation lesion. Thus, the assessment may be performed while the lesion is being created.

Another method is included for forming a transmural lesion from a first surface of a targeted biological tissue to an opposed second surface thereof. The method includes manipulating an antenna assembly of an ablation instrument into engagement with or substantially adjacent to the tissue first surface, and generating an electromagnetic field from the antenna assembly sufficiently strong to cause tissue ablation to the tissue first surface. The method further includes piercing a needle member, having an elongated shaft, into the targeted biological tissue from the tissue first surface. The needle member includes a plurality of needle electrodes spaced-apart along the elongated shaft. Transmitting or receiving electrical signals from one or more needle electrodes is performed to measuring at least one of conduction time, conduction velocity, phase angle, and impedance is performed through the biological tissue at each independent electrode positioned at the respective depth to determine the transmurality of the ablation lesion.

In one specific configuration, the method includes engaging a first side electrode with the tissue first surface at a location radially spaced from a longitudinal axis of the shaft, and engaging a second side electrode with the tissue first surface at a location radially spaced from a longitudinal axis of the shaft, and spaced-apart from the first side electrode. Subsequently, the method includes measuring the at least one of conduction time, conduction velocity, phase angle, and impedance between the two or more needle electrodes and the first side electrode and the second side electrode.

The engaging a first side electrode is performed on one side of the ablation lesion, and the engaging a second side electrode is performed on an opposite side of the ablation lesion. Further, the piercing event includes driving the shaft into the ablation lesion until the first side electrode and the second side electrode engage the first tissue surface.

In yet another aspect of the present, a method for treating medically refractory atrial fibrillation of the heart is provided. This method includes manipulating an antenna assembly of an ablation instrument into engagement with or substantially adjacent to a first surface of targeted cardiac tissue of the heart, and generating an electromagnetic field from the antenna assembly sufficiently strong to cause tissue ablation to the first surface to form an ablation lesion extending from the first surface toward an opposed second surface of the heart. In accordance with this aspect of the present invention, before, during or after generating, the method next includes piercing a needle member having an elongated shaft into the targeted cardiac tissue from the heart first surface. The needle member includes a plurality of needle electrodes spaced apart along the elongated shaft such that when the needle member pierces into the ablation lesion, the electrodes are placed at different respective depths of the cardiac tissue from the tissue first surface to the tissue second surface. Next the method includes transmitting and/or receiving electrical signals to measure at least one of conduction time, conduction velocity, phase angle, and impedance through at least a portion of the targeted cardiac tissue at each independent electrode positioned at the respective depth to determine the transmurality of the ablation lesion. The manipulating, generating, piercing and measuring events are repeated to form a plurality of strategically positioned ablation lesions and/or to divide the left and/or right atria to substantially prevent reentry circuits.

In one specific embodiment, the ablation lesions are strategically formed to create a predetermined conduction pathway between a sinoatrial node and an atrioventricular node of the heart. In another application, the manipulating, generating, piercing and measuring are repeated in a manner isolating the pulmonary veins from the epicardium of the heart.

### BRIEF DESCRIPTION OF THE DRAWINGS

The assembly of the present invention has other objects and features of advantage which will be more readily apparent from the following description of the best mode of carrying out the invention and the appended claims, when taken in conjunction with the accompanying drawing, in which:

FIG. 1 is a fragmentary side elevation view, in cross-section, of a transmurality assessment instrument for assessing the transmurality of an ablation lesion accordance with one embodiment of the present invention.

FIG. 2 is a fragmentary side elevation view, in cross-section, of an alternative embodiment of the transmurality assessment instrument of FIG. 1 having side electrodes.

FIG. 3 is a fragmentary, top perspective view of an ablation assembly of an ablation instrument.

FIG. 4 is an enlarged, fragmentary, side elevation view, in partial cross-section, of the transmurality assessment instrument of FIG. 2.

FIG. 5 is a front elevation view of an alternative embodiment of the transmurality assessment instrument of FIG. 2 mounted to an ablation assembly.

FIG. 6 is a fragmentary, top perspective view, partially cut-away, of another alternative embodiment of the transmurality assessment instrument of FIG. 2 mounted to a guide assembly for a sliding ablation assembly of an ablation instrument.

FIG. 7 is a top perspective view, in cross-section, of an ablation instrument with the transmurality assessment instrument of FIG. 1 engaged against cardiac tissue.

FIGS. 8A and 8B are schematic diagrams of a method to assess the transmurality of an ablation lesion in accordance with the present invention.

### DETAILED DESCRIPTION OF THE INVENTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various Figures.

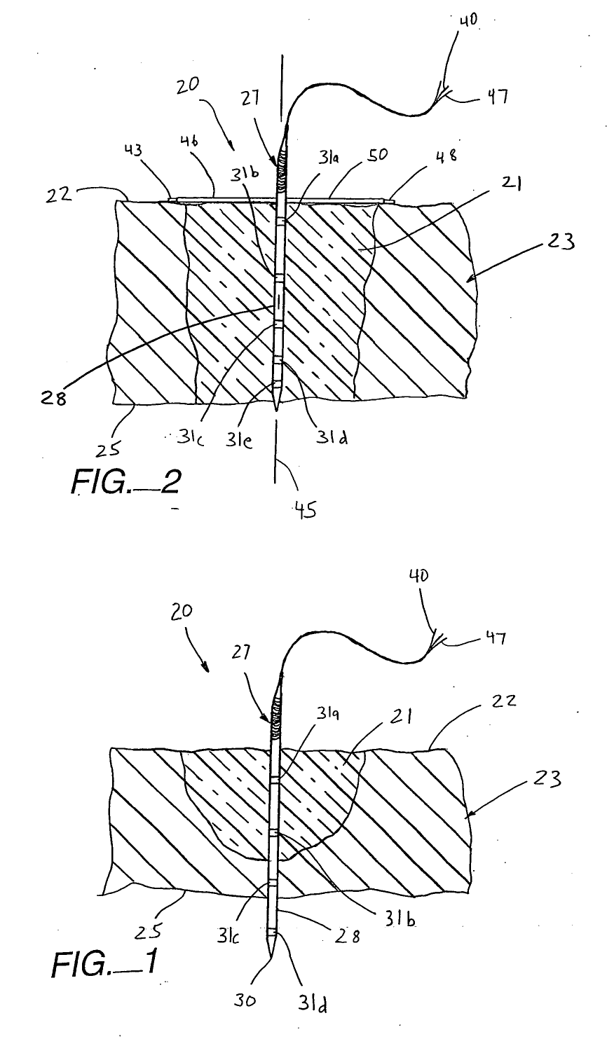

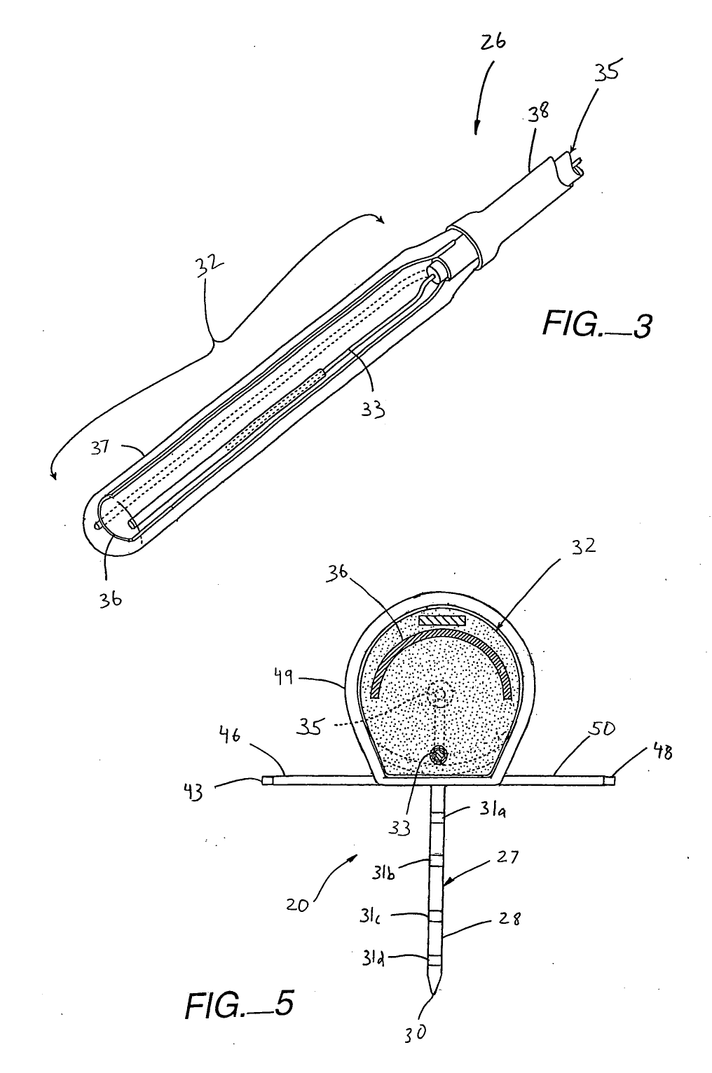

Turning now to FIGS. 1-2, an instrument or device, generally designated 20, is provided to assess the transmurality of an ablation lesion 21 which extends from a first surface 22 of a targeted biological tissue 23 toward an opposed second surface 25 thereof. As will be described in greater detail below, these lesions are generally formed during surgical tissue ablation procedures through the application of tissue ablation instruments 26 (FIGS. 3 and 5-7). These tissue ablation instruments typically ablate tissue through contact with the first surface 22 of the tissue. The present invention, thus, evaluates the effectiveness, depth and completeness (i.e., the transmurality) of the ablation from the first surface toward the second surface.

The measurement instrument 20 includes a needle member, generally designated 27, having an elongated shaft 28 and a distal tip portion 30 adapted to pierce the first surface 22 of the targeted tissue 23. As best viewed in FIG. 1, a plurality of needle sensors 31 are spaced-apart along the elongated shaft 28 so that when the needle member is advanced into lesion 21 from the first surface 22 toward the second surface 25 of the tissue 23, at least two or more of the plurality of needle sensors 31 are positioned at different respective depths of the biological tissue between the tissue first surface to the tissue second surface. Each needle electrode is adapted to selectively transmit and/or receive electrical signals. Through cooperative analysis of the acquired signals transmitted or received by the sensors, the depth and transmurality of the ablation lesion can be determined.

Accordingly, one or more direct measurements of various tissue structure characteristics of the targeted biological tissue can be performed by the sensors 31 positioned at the selected depths rather than a single measurements from sensors placed at the first surface 22 and/or the second surface 25 as practiced by the current systems described in the art. Through comparative analysis of the measured sensor information from two or more of the sensors 31, a more detailed and precise assessment of the depth and/or transmurality of the ablation lesion 21 may be determined. Moreover, as will be described in greater detail below, by selectively and dynamically configuring each needle electrode as either a transmitter or receiver of electrical signals during an ablation procedure, the present invention can be utilized to obtain more specific and detailed information regarding one or more lesion characteristics. This is very advantageous since the precise depth or progression of the lesion can be directly measured during or after creation of the ablation, via the corresponding sensor, especially in locations where the biological tissue is relatively thick and difficult to access (e.g., dome of the left atrium, intra-atrial groove, or the pulmonary veins of a heart). Moreover, the sensors can be applied to assess the thickness of the tissue by determining which ones are surrounded by biological tissue, and which sensors are surrounded by fluid (e.g., blood), as will be discussed in greater detail below.

Briefly, the present invention is suitable for use in connection with tissue ablation instruments adapted to ablate the biological tissue walls of internal organs and the like. These tissue walls typically have wall thickness from one surface of the tissue to an opposite surface of the tissue in the range of about 2 mm to about 10 mm. Thus, through direct contact with or exposure of the one surface of the tissue to an ablation assembly 32 of the ablation instrument 26, the formation of the ablation lesion generally propagates from the one surface toward the opposed second surface of the tissue. It will be understood, however, and as set forth below, that any modality of ablative energy may be applied.

As shown in FIG. 3, these tissue ablation instruments 26 typically include a distal, ablation assembly 32 which emits ablative energy in a manner sufficient to cause tissue ablation. Thus, by manipulating and strategically placing the ablation assembly 32 adjacent to or in contact with the targeted biological tissue to be ablated, strategic lesion formation can occur. By way of example and as will be described in greater detail below, a series of strategically placed ablation lesions around heart collectively create a predetermined conduction pathway. More specifically, the conduction pathway is formed between a sinoatrial node and an atrioventricular node of the heart, such as required in the MAZE III procedure to treat arrthymias.

Any source of ablative energy may be employed to achieve ablation. These include, but are not limited to, Radio Frequency (RF), laser, cryogenic, ultrasound, one or more resistive heating elements, microwave, or any other energy which can be controllably deployed to ablate tissue. The source of ablation can also be one or a family of chemical agents. For example, localized ethanol injection can be used to produce the ablation lines. RF probes that apply an RF conduction current in the range of about 450 kHz to about 550 kHz. Typical of these RF ablation instruments include ring electrodes, coiled electrodes or saline electrodes. Another source of ablative energy are laser based energy sources sufficient to ablate tissue. These include CO₂ or Nd: YAG lasers which are transmitted to the ablation assembly 32 through fiber optic cable or the like. Yet another alternative energy source is cryogenic energy. These cryogenic probes typically apply a cryogenic fluid, such as a pressurized gas (e.g., Freon), through an inflow lumen to a decompression chamber in the ablation assembly. Upon decompression or expansion of the pressurized gas, the temperature of the ablation assembly is sufficiently reduced to cause tissue ablation upon contact therewith. The ablative energy may also be ultrasoncially based. For example, one or a series of piezoelectric transducers may be provided as an ablative element which delivers acoustic waves sufficient to ablate tissue. Such transducers include piezoelectric materials such as quartz, barium oxides, etc.

One particularly effective source of ablative energy, however, is microwave energy which is emitted as an electromagnetic field by the ablation assembly. One advantage of microwave energy, as mentioned, is that the field is easier to control and safer than direct current applications. Typically, the microwave energy permeates the tissue to a depth proportional to the energy applied. The microwave probes, further, are capable of generating substantially larger and longer lesions than RF catheters, which greatly simplifies the actual ablation procedures. Moreover, recent advances in the antenna assembly designs enable even greater control of the field emission in predetermined directions for strategic lesion formation.

Briefly, referring back to FIG. 3, an ablation instrument 26 is shown having an ablation assembly 32 adapted to ablate the targeted tissue. More specifically, the ablation assembly 32 generally includes an elongated antenna 33 coupled to a transmission line 35 for radially generating the electric field substantially along the longitudinal length thereof. To directionally control the radiation of ablative energy, a shield device 36 substantially shields a surrounding radial area of the antenna wire 33 from the electric field radially generated therefrom, while permitting a majority of the field to be directed generally in a predetermined direction. An insulator 37 is disposed between the shield device 36 and the antenna 33, and enable the transmission of the directed electric field in the predetermined direction.

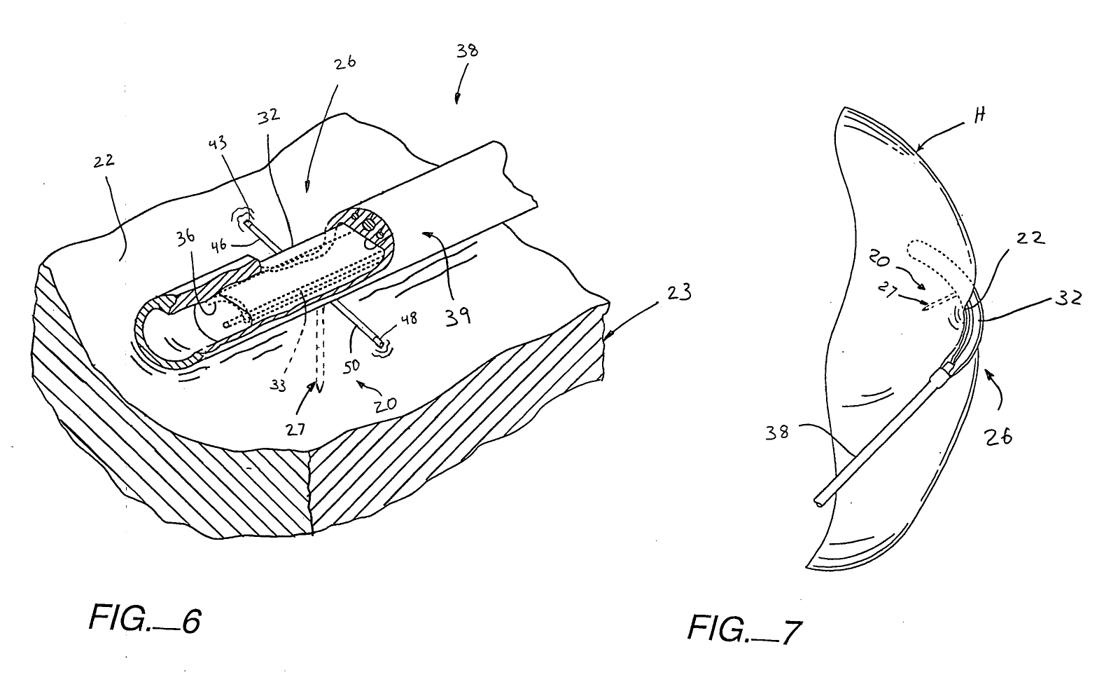

The ablation instrument 26 includes a manipulating device 38 which cooperates with the ablation assembly 32 to orient the antenna and shield device in position to perform the desired ablation. This manipulating device 38, for example, may include a handle member or the like coupled to the ablation assembly, as shown in FIGS. 3 and 7. Another example of the manipulating device 38 includes a guide assembly 39 of FIG. 6, having a track system slideably receiving the ablation assembly 32. Such microwave ablation systems are described in the U.S. Pat. Nos. 6,245,062; 6,312,427 and 6,287,302 to Berube et al.; U.S. patent application Ser. No. 09/484,548 to Gauthier et al., filed Jan. 18, 2000, and entitled “MICROWAVE ABLATION INSTRUMENT WITH FLEXIBLE ANTENNA ASSEMBLY AND METHOD”, and U.S. patent application Ser. No. 09/751,472 to Mody et al., filed Dec. 29, 2000, and entitled “A PREFORMED GUIDE APPARATUS WITH A SLIDING MICROWAVE ABLATION INSTRUMENT AND METHOD”, each of which is incorporated herein by reference.

Briefly, when microwave energy is applied, the power supply (not shown) will include a microwave generator which may take any conventional form. The optimal frequencies are generally in the neighborhood of the optimal frequency for heating water. By way of example, frequencies in the range of approximately 800 MHz to 6 GHz work well. Currently, the frequencies that are approved by the Federal Communication Commission (FCC) for Industrial, Scientific and Medical work includes 915 MHz and 2.45 GHz and 5.8 GHz (ISM band). Therefore, a power supply having the capacity to generate microwave energy at frequencies in the neighborhood of 2.45 GHz may be chosen. A conventional magnetron of the type commonly used in microwave ovens is utilized as the generator. A solid-state amplifier could also be used. It should be appreciated, however, that any other suitable microwave power source (like a Klystron or a traveling-wave tube (TWT)) could be substituted in its place, and that the explained concepts may be applied at other frequencies like about 434 MHz, 915 MHz or 5.8 GHz (ISM band).

Referring back to FIGS. 1 and 2, the plurality of spaced-apart sensors 31 enable direct measurement of the tissue properties at their respective depths to assess ablation transmurality. Preferably, the sensors can selectively transmit and/or receive electrical signals to measure at least one of the conduction time, the conduction velocity, the phase angle, and the impedance through at least a portion of the targeted tissue, at their respective depths, to determine the transmurality of the ablation lesion. As mentioned above, such measured electrical characteristics have been found to indicate the viability or non-viability of the tissue.

In accordance with one specific embodiment of the present invention, each needle sensor 31 is provided by a needle electrode which is selectively adapted to transmit and/or receive electrical signals. Thus, the electrodes typically operate in a coordinated fashion to transmit and/or receive the electrical signals across the tissue region being measured. When an associated needle electrode 31 is selected to transmit electrical signals, this transmitting electrode is coupled to a signal generating source, such as a standard function generator readily available. n contrast, when the associated needle electrode 31 is selected to receive electrical signals, this receiving electrode is coupled to a receiving unit, such as a multimeter or other suitable acquisition system. Accordingly, the electrical signals propagate through selected portions of the biological tissue between the corresponding needle electrodes 31 at the respective depths to determine transmurality of these portions of the tissue.

For instance, for the measurement instrument 20 of FIG. 1 having four needle electrodes 31a-31d, any combination of signal propagation may be performed between the associated needle electrodes (i.e., 31a and 31c, 31a and 31d , 31a and 31b, 31b and 31c, 31b and 31d, or 31c and 31d). This versatility, of course, enables a more precise analysis of the targeted tissue region for greater control of the ablation instrument.

Preferably, these needle electrodes 31 are provided by ring electrodes longitudinally spaced-apart along the shaft 28. Such electrodes may be composed of a conductive or metallic material, such as silver, platinum or other bio-compatible metals suitable for the purposes described herein. Non-metallic conductive electrodes like Ag-AgCl, or saline electrodes could also be used.

Alternatively, rather than provide a continuous ring extending annularly around the shaft, each needle electrode at a respective depth may be comprised of a plurality of smaller electrode components (not shown) annularly extending around the shaft which are commonly connected. Moreover, while the longitudinal spacing between the adjacent needle electrodes are illustrated as generally equal, the spacings may vary depending upon their proposed function. For example, as shown in FIG. 2, the spacing between adjacent needle electrodes 31a and 31b near the proximal end of the shaft 28 may be greater than the spacing between the adjacent needle electrodes 31d and 31e near a distal end of the shaft. In this manner, since the data measured from the distal ring electrodes, which are positioned more toward the opposed second surface side of the tissue, may be more critical to assess the state of transmurality, the closer needle electrode spacings can provide a more detailed analysis. Comparatively, the measurements conducted by the electrodes at the proximal end of the shaft have a substantially greater probability of being sufficiently ablated since this tissue is closer to the ablative energy source.

By way of example, for a needle member having a shaft in the range of about 2 mm to about 15 mm in length, and with a diameter in the range of about 0.7 mm to about 1.2 mm, and more preferably about 1.0 mm, the spacing between the adjacent electrodes at the proximal portion of the shaft may be in the range of about 1 mm to about 5 mm while the spacing between the adjacent electrodes at the distal portion of the shaft may be in the range of about 1 mm to less than 1 mm. Comparatively, for a similarly dimensioned shaft 28 where the ring electrodes are equally spaced, the adjacent electrodes 31 may be in the range of less than 1 mm to about 5 mm apart. The needle electrode spacing, of course, can be configured to best accommodate the anticipated targeted tissue to be ablated (i.e., the anticipated thickness of the tissue). As noted, typical cardiac tissue is about 4 mm to about 6 mm deep. The smaller the spacing between the adjacent electrodes, the greater the resolution. This also pertains to the height of the electrodes in that the smaller the height, the greater then number of electrodes that can be placed along the needle shaft 28.

Each needle electrode 31 is coupled to a respective transmission line 40 to transmit the received signal to a processing unit (to be discussed in greater detail below). Accordingly, referring now to FIG. 4, the shaft 28 of the needle member 27 preferably includes a passage or lumen 41 sized for receipt of the transmission lines 40 coupled to each needle electrode from a backside thereof. As mentioned, depending upon the application of the selected ring electrode 31, the proximal end of the corresponding transmission line 40 may be coupled to an electrical signal generator or receiver, or otherwise a data acquisition system able to send a desired input signal from any one or more electrodes and selectively acquire data received from any one or more electrodes in response to the input (either of which are not shown).

The shaft 28 may be grooved or define a plurality of annular slots 42 formed and dimensioned for receipt of the ring-electrodes therein. Preferably, the width and depth of each slot 42 is substantially similar to that of the respective ring electrode 31 so that it may be seated generally flush with the exterior surface of the shaft, and substantially free of gaps or spaces. This would facilitate smooth insertion and advancement of the shaft 28 into the targeted tissue 23 once the distal tip portion 30 pierces the first surface 22 thereof.

To prevent signal interference between adjacent ring electrodes 31, the shaft 28 is preferably composed of a non-conductive, bio-compatible material, or otherwise is adapted to electrically isolate the electrodes 31. This electrical isolation between the adjacent needle electrodes further enables closer spacing therebetween. Such materials includes ceramics, plastics, or any other suitable materials having similar isolating characteristics. It will be appreciated, however, that electrical isolation between adjacent electrodes may also be provided by an insulator therebetween, as well.

In another specific embodiment of the present invention, the measurement instrument 20 includes a first side electrode 43 which is adapted to be positioned in contact with the tissue surface 22 when the transmurality measurement instrument is fully advanced into the targeted tissue. As best viewed in FIG. 2, this side electrode 43 is preferably placed to contact the first surface 22 at a location spaced-apart from a longitudinal axis 45 of the shaft 28. Similar to the needle electrodes 31, this side electrode 43 is adapted to selectively transmit and/or receive electrical signals between the first surface of the biological tissue and one or more of the needle electrodes 31 advanced in the targeted biological tissue 23. Accordingly, the electrical signals will generally propagate across a greater portion of the targeted biological tissue, as compared to only between the needle electrodes 31 themselves.

The first side electrode 43 is preferably affixed at a position, relative to the ring electrodes 31, so that the distance between the first side electrode and the ring electrodes remains relatively fixed during operation. Thus, by positioning the first side electrode 43 at the proximal portion of shaft 28, although radially spaced from the shaft longitudinal axis, the distance between the first side electrode and each successive ring-electrode 31 successively increases (FIG. 2). Depending upon the electrical characteristic being measured across the targeted tissue, the known distances therebetween become more relevant, and factor into the determination. For example, when measuring certain tissue characteristics to determine the transmurality of the lesion, the distance between the electrodes may be required for the calculation.

When the needle member 27 pierces the ablation lesion 21, the first side electrode 43 is positioned, relative the longitudinal axis 45 of the shaft 28, to contact the first tissue surface 22 at a location along one side of the longitudinal axis of the ablation lesion 21. More preferably, the distance of the first side electrode 43 from the longitudinal axis of the lesion (and from the longitudinal axis of the needle member 27, is sufficient to place the electrode just inside the edge of the ablation lesion 21 to contact viable tissue, the acquired signal will be a function of the lesion development through the depth of the tissue rather than through the lateral lesion development. By way of example, the side electrode 43 is spaced from longitudinal axis of the shaft 28 in the range of about 1 mm to about 10 mm. It will be appreciated, however, that the electrodes can be placed just outside of, or directly at, the edge of the lesion as well, and that this distance between the electrode and the longitudinal axis of the shaft may be adjusted depending upon the anticipated width of the lesion.

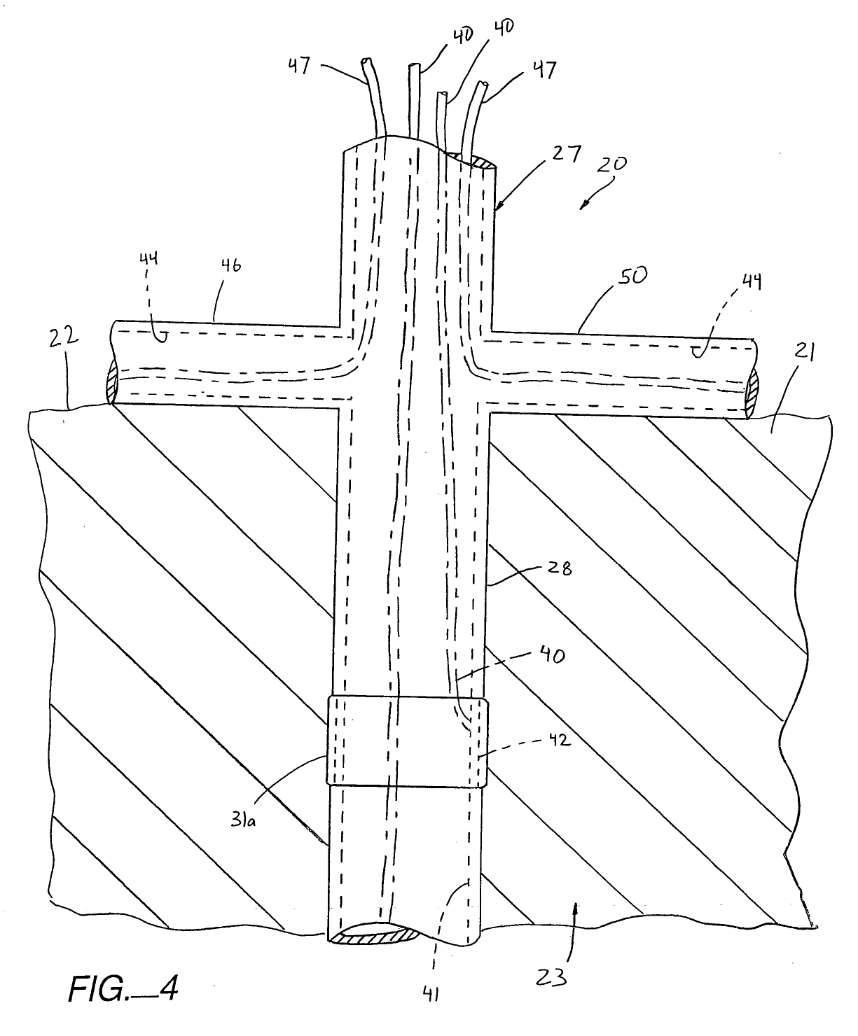

To support the first side electrode 43 at a fixed location relative the needle electrodes 31, in one specific embodiment, a first support member 46 extends radially away from the longitudinal axis of the shaft 28. The first support member 46 is substantially rigid, in one embodiment, and includes a proximal end integrally mounted to the proximal portion of the shaft 28. While the first support member 46 is illustrated at an orientation extending substantially perpendicular to the longitudinal axis of the shaft, it will be appreciated that the entire support member, or at least a distal end portion thereof, may be slightly angled downwardly to facilitate contact with the first tissue surface 22 when the shaft is penetration the tissue. In this configuration, the support member 46 may be slightly flexible, although resilient, to prevent penetration of the first support member into the tissue. The first support member 46, further, will be oriented to position the first side electrode 43 outside of the ablation lesion 21. For example, in one configuration where the measurement instrument 20 is mounted to the ablation assembly, as an integral unit (to be discussed and as shown in FIGS. 5-7), the first support member 46 extends outwardly in a direction generally perpendicular to the longitudinal axis of the ablation assembly 32 of the ablation instrument 26.

The first side electrode 43 is preferably affixed to the distal end of the first support member 46, but may also be affixed to an underside thereof. The side electrode 43 can also cover the entire length of the first support member 46. Thus, when the needle member 27 is fully advanced into the targeted tissue, contact with the first tissue surface 22 by the side electrode will be assured. A lumen extends longitudinally through the support member for receipt of a transmission line 47 coupled to a backside of the first side electrode 43. This transmission line, depending upon the application, is coupled to the transmission source which generates the appropriate signals.

In still another specific embodiment, another selectable electrical signal transmission and/or receiving source is provided by a second side electrode 48. As shown in FIG. 2, this second electrode is adapted to contact the first tissue surface 22 at a location also spaced-apart from the longitudinal axis of the shaft 28, but on a side opposite the first side electrode 43. Thus, when the needle member 27 pierces the ablation lesion 21, the first side electrode 43 is oriented to contact the first tissue surface 22 at a location along one side of the longitudinal axis of the ablation lesion 21 while the second side electrode 48 contacts the first tissue surface 22 at a location along the other side of the longitudinal axis of the ablation lesion 21. In this manner, the propagation of electrical signals can be performed from both sides of the ablation lesion between the side electrodes and the needle electrodes. This is advantageous in that the tissue characteristics can be measured between side electrodes as further evidence of transmurality, such as conduction time, conduction velocity, phase angle, and impedance through at least a portion of the targeted tissue, at the respective depth, to determine the transmurality of the ablation lesion created or being created therein.

Similar to the first side electrode 43, a second support member 50 extends radially away from the longitudinal axis of the shaft 28 preferably at the same longitudinal position along the shaft 28. FIGS. 2, 5 and 6 best illustrate that the second support member extends radially from the shaft 28 in a direction about 180° from the first support member 46. The other physical characteristics of the second side electrode 48 and the associated second support member 50 are similar to the first side electrode 43 and the first support member 46. Thus, the second side electrode and associated support member will not again be discussed in detail.

Further, while the first and second side electrodes 43, 48 have been described and illustrated as integrally mounted to the needle member as a single unit, it will be appreciated that the side electrodes can be independent of the needle member, and can be independently placed into contact with the tissue surface. The side electrodes 43, 48 can then be positioned at varying distances from the longitudinal axis of the shaft 28, as well as enable contact with the tissue surface in regions which might not otherwise allow contact due to the topography thereof.

As above-indicated, the needle member 27 of the instrument may be advanced into the targeted tissue before, during or after creation of the ablation lesion by a tissue ablation instrument. One advantage of monitoring the ablation during creation thereof, for instance is that the formation of the ablation lesion can be monitored in real time between the needle electrodes 31.

Moreover, the measurement instrument 20 of the present invention may be mounted directly to or integrally formed with the ablation assembly 32. In this instance, mounting structure 49 may be included which enables removable mounting of the needle member 27 to the ablation assembly 32, or the measurement instrument may be integrally formed with the ablation instrument 26. Referring back to FIGS. 5 and 6, the needle member 27 is illustrated fixedly mounted to an underside of the ablation assembly, extending in a direction proximate that of the predetermined direction of the emitted ablative energy.

Accordingly, in the embodiment of FIG. 5, when the ablation assembly 32 of the ablation instrument 26 is strategically placed along and downwardly adjacent to the targeted tissue 23, the needle member 27 penetrates the first tissue surface 22 and advances into the targeted tissue 23 proximate the antenna assembly. The configuration of FIG. 6, on the other hand, illustrates that the needle member 27 may be mounted to the guide assembly 39. Thus, when this assembly is oriented and placed along the tissue surface, the needle member 27 also penetrates the first tissue surface 22 and advances into the targeted tissue 23. In either embodiment, the formation of ablation lesion may be monitored from the commencement of the ablation formation to assess transmurality. Moreover, the needle member 27 functions as a partial anchor device to secure the ablation assembly 32 or the guide assembly 39 proximate to the targeted tissue.

In these configurations, as illustrated, the support members 46, 50, supporting the associated side electrodes 43, 48 may extend outwardly from the antenna assembly or the guide assembly, as opposed to the needle member. In other embodiments, the side electrodes may not be integral with the antenna assembly or the needle member. In still other configurations, the guide assembly 39 may include a plurality of needle members (not shown) which function to measure the transmurality of the lesion formation, as well as provide a more definitive anchoring device to the tissue surface.

Additionally, the needle member 27 may be adapted to translate in a lumen of the instrument (not shown), a lumen as part of the antenna assembly or guide assembly for example. The proximal end of the needle member 27, in one configuration, can be mechanically interfaced, or otherwise attached, to an elongated member which, in turn, is operably attached to the handle of the medical ablation instrument. Once the distal end of the ablation assembly is placed upon or proximate a target tissue site, the user can operate a movement control, as part of the handle, translating the needle member towards the first surface 22 of the target tissue 23. The lumen is configured to deflect the needle member, encouraging the needle member to engage and advance into the target tissue 23 at a predetermined angle with the target tissue 23 first surface 22, preferably within ±45° and more preferably about 90°. Once the transmurality of the ablation is complete, the user can then operate the movement control to retract the needle member back within the lumen.

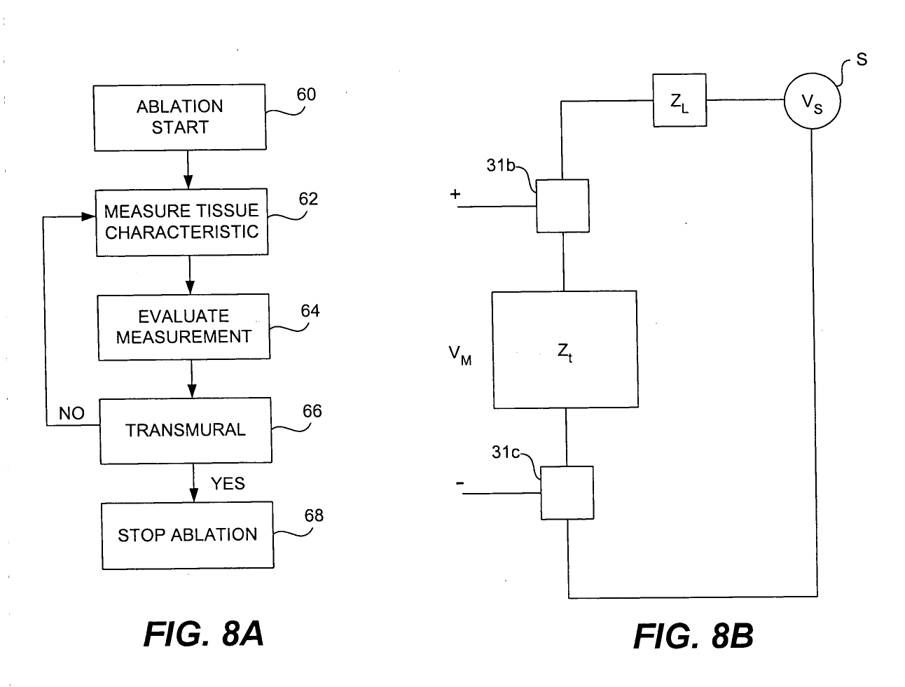

Now briefly referring also to FIG. 8, a general methodology, in accordance with the present invention, will be described in greater detail. More specifically, FIG. 8A depicts a flow chart of steps to assess the progression of an ablation lesion through targeted tissue, ultimately determining when the lesion is transmural. In a first step 60, the ablation process is initiated with the application of ablative energy directed toward the targeted tissue 23. During the ablation procedure, the tissue characteristic is then measured and evaluated in steps 62 and 64, respectively. These measured characteristics are related to at least one of the conduction time, the conduction velocity, the phase angle, and the impedance of the targeted tissue. Based upon these measurements a transmural assessment is made in a step 66. If the conclusion of the assessment is that transmurality is not achieved, control is directed back to the tissue characteristic measurement step 62. However, if transmurality is achieved, the ablation process is stopped in a step 68. The steps of FIG. 8A may be performed by a User, a surgeon for example, or may be performed as part of a program executed by a central processing unit.

It is important to note that the transmural assessment procedure of FIG. 8A can be performed between any two measurement elements, various electrodes for example, as described herein. Therefore, as should be readily apparent, the assessment procedure may be repeated for a series of measurement element pairs, each sub-procedure resulting in a partial transmurality assessment, all the sub-procedures collectively resulting in the transmurality assessment of the ablation lesion itself.

Referring also to FIG. 8B, an example tissue measurement setup will be described in greater detail. FIG. 8B depicts an exemplary setup for the measurement of tissue impedance through a portion of biological tissue between two measurement elements, sensors 31b and 31c from the device of FIG. 5 from the device of FIG. 5 in this example. As shown, a source S is electrically connected to sensor 31b. The source signal Vₛ is applied to sensor 31b through a known load impedance Z_{L}. The source signal Vs propagates through a portion of target tissue between sensors 31b and 31c, the target tissue having an impedance Z_{T}. During the step of measuring tissue impedance 62, the voltage difference V_{M} between sensors 31b and 31c is measured. Since the impedances Z_{L} and Z_{T} form a simple voltage divider, the tissue impedance Z_{T} can be calculated from the measured voltage V_{M}.

The impedance Z_{T} measurement is then evaluated in the step 64. More specifically, as depicted in FIG. 1, as the ablation propagates through the tissue, from sensor 31b toward sensor 31c, the impedance is observed to change with respect to previously obtained values, generally decreasing in value over time. Once the ablation propagates past sensor 31c toward sensor 31d, the impedance measured in step 62 between sensors 31b and sensor 31c, as compared with previous measurements, is observed to be constant. The constant measurement of impedance will be evaluated in step 64, the result of the sub-procedure being that the ablation is transmural with respect to sensor 31b and sensor 31c.

It should be apparent that the determination of the ‘constant measurement’ may be predetermined as being something other than equal, with respect to previous measurements. For example, when the impedance change is noted to be within a certain limit, the change in value may be deemed constant. Additionally, the sampling time associated with the assessment loop steps 62, 64, and 66 may be any suitable time, preferably to minimize the time in determining transmurality. Alternatively, the assessment loop sampling time may be directly proportional to the acquired assessment value itself, the change in impedance for example. When a large change in value is observed, less sampling is required, and when there is a small change in value observed the sampling rate may be increased to better determine the exact time of transmurality.

The evaluation step 64 may also include a conditioning step, dependent on the specific measurement made. For example, the signal representative of the tissue characteristic measurement may be filtered to remove unwanted signals which make evaluation of the measurement more difficult. These unwanted signals may be derived from inconsistent contact between the measurement elements of the measurement instrument 20 and the tissue 23 or from an input signal provided as part of the tissue characteristic measurement.

It should be readily understood that the transmitted signals are selected, or otherwise defined, based upon the desired tissue measurement. For example, certain transmitted signals may be designed to passively interface with the tissue, while other signals may be designed to induce a response from the tissue itself. Passively, as used in the immediate discussion, means that the transmitted signals do not interfere with the normal rhythm of the heart.

For example, any two sensors 31 may be configured to passively measure the electrical impedance therebetween. This measurement can be made using any suitable method, simple utilization of a standard ohmmeter for example. However, an electrical circuit such as the setup of FIG. 8B is preferred. The source signal Vₛ may be any suitable passive voltage at a frequency of at least 100 khz, preferably five volts ac at a frequency of 100 khz, more preferably, at a frequency of 500 khz. It is important to note that the source may be selected to also carry out the ablation process as well as provide excitation for the tissue characteristic measurement.

Alternatively, two or more electrical sensors 31, 43, 48 (as shown in FIG. 5, and as will be described below) may be configured to transmit and receive electrical signals, the transmitted signal intended to induce a response from the cardiac tissue. For example, Sensor 43 may be configured as a pacing electrode adapted to transmit an electrical signal and sensor 48 may be a recording electrode adapted to recording the electrical response to the transmitted signal. Upon the initiation of an electrical signal via sensor 43, a response is generated in the cardiac tissue, such response being received by sensor 48.

Since ablated tissue will not transmit the signal therethrough, a signal delay will be observed, in the tissue characteristic measurement made in the step 62 of FIG. 8A, between sensor 43 and sensor 48, the delay being directly related to ablation depth. As with the impedance measurement described above, when the tissue characteristic measurement (delay) is observed as being constant over a predetermined period of time, the ablation depth will be maximized, transmurality relative to the current ablation lesion being created for example.

While the above example is provide with respect to side electrodes or sensors 43 and 48, the example is applicable with respect to any sensor pair 31, 43, 48. For example, the sensor 43 may be configured to provide a pacing signal and one or more sensors 31 may be configured as recording electrodes. In this example, however, the evaluation step 64 of FIG. 8A would be based on perceiving a signal received at any particular sensor 31. More specifically, with reference back to FIG. 1, as the ablation lesion propagates through the tissue 23 toward the second surface 22 passing by sensor 31b, sensor 31b will no longer be able to receive the pacing signal, the ablated tissue being unable to electrically transmit the pacing signal. Therefore, when a signal in response to the pacing signal is no longer received at sensor 31b the ablation lesion has propagated passed sensor 31b toward second tissue surface 25. At this time the sensor 31c may be the recording sensor of interest until it no longer receives a responsive signal. This process is continued for remaining sensors 31 until tissue transmurality is achieved.

The number of sensors 31 may be greater than those shown in FIGS. 1 and 2, and may be more closely positioned as generally shown in FIG. 2, to better assess transmurality. Alternatively, the final assessment of transmurality for this example may be calculated based upon a rate of ablation noted during the assessment steps 62-66 of FIG. 8A. While the rate of ablation through the tissue 23 toward second surface 25 typically decreases during the ablation process, this rate can be calculated by observing when the responsive signal is no longer received by sensors 31a, 31b, . . . , 31n. Therefore, even though a sensor 31 may not exist proximate or next to the tissue surface 25, transmurality can still be assessed. It should be readily understood that a sensor 31 which exists outside of the tissue 23, past tissue surface 25, would clearly be ascertained as being outside tissue 23 since a signal responsive to the pacing signal transmitted would not be received at that sensor 31.

One significant application of the present invention is in the treatment of medically refractory atrial fibrillation of the heart. For example, as represented in FIG. 7, an ablation instrument 26 can be manipulated to position the ablation assembly 32 into engagement with or substantially adjacent to the epicardium or endocardium of the targeted cardiac tissue 23 of the heart H. Ablation energy, preferably an electromagnetic field, is generated from the ablation assembly 32 sufficiently strong to cause tissue ablation to form an elongated ablation lesion 21 extending from the first surface toward an opposed second surface 25 of the heart. Before, during or after the transmission of ablation energy from the ablation assembly 32, the shaft 28 of the needle member 27 of the measuring instrument is introduced into the targeted cardiac tissue from the heart first surface 22. As viewed in FIGS. 1, 2 and 5, the needle member 27 includes a plurality of needle electrodes 31 spaced apart along the elongated shaft 28. These electrodes, as mentioned, are adapted to selectively transmit and/or receive electrical signals from one or more electrodes 31 to measure at least one of conduction time, conduction velocity, phase angle, and impedance through at least a portion of the targeted cardiac tissue. This data, of course, is applied to determine the completion or transmurality of the ablation lesion 21 created or being created therein. To fully treat the medically refractory atrial fibrillation, the procedures are repeated (i.e., the manipulating, generating, piercing and transmitting or receiving) to form a plurality of strategically positioned ablation lesions and/or to divide the left and/or right atria to substantially prevent reentry circuits.

For instance, using this technique, the pulmonary veins may be electrically isolated from other tissues of the heart. In particular, the strategic positioning of the ablation lesions (not shown) cooperates to create a predetermined conduction pathway between a sinoatrial node and an atrioventricular node of the heart. Further, this procedure may be performed during open or minimally invasive surgical procedures. In the latter procedure, the heart may be beating or arrested.

In another specific embodiment, a first side electrode 43 may be engaged with the tissue first surface 22 of the heart at a location radially spaced from a longitudinal axis of the shaft 28. Similarly, a second side electrode 48 is engaged with the tissue first surface at a location radially spaced from a longitudinal axis of the shaft, and spaced-apart from the first side electrode. Both the first side electrode and the second side electrode are preferably supported by a first support member 46 and a second support member 50, respectively, each extending radially away from the longitudinal axis of the shaft 28. The first support member 46 is adapted to support the first side electrode 43 and the second support member 50 is adapted to support the second side electrode 48, both at positions radially spaced from shaft longitudinal axis. Thus, once the needle member 27 is pierced into the targeted tissue 23, it is advanced until the first side electrode 43 and the second side electrode 48 engages the first tissue surface.

Preferably, the measuring instrument is oriented so that engagement of the first side electrode 43 is performed on one side of the elongated ablation lesion, while the engagement of the second side electrode 48 is performed on an opposite side of the elongated ablation lesion. Applying these two side electrodes, or even just one in some instances, at least one of conduction time, conduction velocity, phase angle, and impedance may be measured across the target cardiac tissue with respect to one or more needle electrodes 31.

1. A device for assessing the transmurality of an elongated ablation lesion from a first surface of a targeted biological tissue to an opposed second surface thereof comprising:
a needle member having an elongated shaft and a distal tip portion adapted to pierce the tissue first surface and advance into the targeted biological tissue; and
at least two needle electrodes spaced apart along the elongated shaft, each said electrode being adapted to selectively transmit or receive electrical signals to measure at least one of conduction time, conduction velocity, phase angle, and impedance through at least a portion of the targeted biological tissue, to determine the transmurality of an ablation lesion created therein.

2. The device according to claim 1, wherein
said needle electrodes include at least three electrodes.

3. The device according to claim 2, wherein
said needle electrodes are evenly spaced apart along the shaft.

4. The device according to claim 3, wherein
said needle electrodes are spaced apart in the range of about less than 1 mm to about 5 mm.

5. The device according to claim 2, wherein
the spacings between adjacent needle electrodes are less at a distal portion of the shaft than between adjacent needle electrodes at a proximal portion of the shaft.

6. The device according to claim 1, wherein
said needle electrodes are ring electrodes.

7. The device according to claim 1, further including:
a first side electrode adapted to engage the tissue first surface at a location radially spaced from a longitudinal axis of the shaft to measure at least one of conduction time, conduction velocity, phase angle, and impedance with respect to one or more needle electrodes.

8. The device according to claim 7, wherein
said first side electrode is sufficiently radially spaced from the longitudinal axis of the shaft for positioning on one side of the elongated ablation lesion.

9. The device according to claim 7, further including:
a first support member extending radially away from the longitudinal axis of said shaft, and adapted to support said first side electrode such that when said shaft of the needle member is extended into the ablation lesion to position each needle electrode at the different respective depths of the biological tissue, said first side electrode engages the tissue first surface.

10. The device according to claim 9, wherein
said first support member is coupled to the shaft proximate a proximal portion thereof.

11. The device according to claim 9, wherein
said first support member extends substantially perpendicularly away from the longitudinal axis of said shaft.

12. The device according to claim 9, wherein
said first side electrode is mounted proximate a distal tip portion of the first support member.

13. The device according to claim 9, wherein
said first support member is substantially rigid.

14. The device according to claim 7, further including:
a second side electrode adapted to engage the tissue first surface at a location radially spaced from a longitudinal axis of the shaft, and spaced-apart from the first side electrode, to measure at least one of conduction time, conduction velocity, phase angle, and impedance with respect to one or more needle electrodes.

15. The device according to claim 14, further including:
a first support member and a second support member each extending away from the longitudinal axis of said shaft, said first support member being adapted to support said first side electrode and said second support member being adapted to support said second side electrode such that when said shaft of the needle member is extended into the ablation lesion to position each needle electrode at the different respective depths of the biological tissue, at least one of said first side electrode and said second side electrode engages the tissue first surface.

16. The device according to claim 15, wherein
the proximal portions of said first support member and said second support member are mounted to shaft about 180° apart from one another along the longitudinal axis of the shaft.

17. The device according to claim 16, wherein
the proximal portions of the first support member and the second support member are coupled to the shaft proximate a proximal portion thereof.

18. The device according to claim 16, wherein
said first support member and said second support member each extends substantially perpendicularly away from the longitudinal axis of said shaft.

19. The device according to claim 15, wherein
said first side electrode is mounted proximate a distal tip portion of the first support member, and
said second side electrode is mounted proximate a distal tip portion of the second support member.

20. The device according to claim 19, wherein
said first side electrode is sufficiently radially spaced from the longitudinal axis of the shaft for positioning on one side of the elongated ablation lesion, and
said second side electrode is sufficiently radially spaced from the longitudinal axis of the shaft for positioning on an opposite side of the elongated ablation lesion.

21. A method of assessing the transmurality of an elongated ablation lesion from a first surface of a targeted biological tissue to an opposed second surface thereof, said method comprising
before, during or after the creation of the ablation lesion, piercing a needle member having an elongated shaft into the targeted biological from the tissue first surface, said needle member including a plurality of needle electrodes spaced apart along the elongated shaft; and
selectively transmitting or receiving electrical signals from at least two needle electrodes to measure at least one of conduction time, conduction velocity, phase angle, and impedance through at least a portion of the targeted biological tissue to determine the transmurality of the ablation lesion created or being created therein.

22. The method of claim 21, further including:
engaging a first side electrode with the tissue first surface at a location radially spaced from a longitudinal axis of the shaft; and
measuring at least one of conduction time, conduction velocity, phase angle, and impedance with respect to one or more needle electrodes.

23. The method of claim 22, further including:
analyzing the measured data of the at least one of conduction time, conduction velocity, phase angle, and impedance with respect to the one or more needle electrodes to determine the degree of tissue ablation.

24. The method of claim 23, wherein
said needle member includes a first support member extending radially away from the longitudinal axis of said shaft, and adapted to support said first side electrode,
said piercing includes extending the shaft into the ablation lesion until the first side electrode engages the first tissue surface.

25. The method of claim 22, further including:
engaging a second side electrode with the tissue first surface at a location radially spaced from a longitudinal axis of the shaft, and spaced-apart from the first side electrode, and
measuring the at least one of conduction time, conduction velocity, phase angle, and impedance with respect to the one or more needle electrodes.

26. The method of claim 25, wherein
said engaging a first side electrode is performed on one side of the elongated ablation lesion, and
said engaging a second side electrode is performed on an opposite side of the elongated ablation lesion.

27. The method of claim 26, further including:
analyzing the measured data of the at least one of conduction time, conduction velocity, phase angle, and impedance with respect to the one or more needle electrodes to determine the degree of tissue ablation.

28. The method of claim 27, wherein
said needle member includes a first support member and a second support member each extending radially away from the longitudinal axis of said shaft, said first support member being adapted to support said first side electrode and said second support member being adapted to support said second side electrode, and
said piercing includes extending the shaft into the ablation lesion until the first side electrode and the second side electrode engages the first tissue surface.

29. The method of claim 28, wherein
the proximal portions of said first support member and said second support member are mounted to shaft about 180° apart from one another along the longitudinal axis of the shaft.

30. A tissue ablation assembly adapted to ablate a targeted biological tissue from a first surface thereof to an opposed second surface thereof to form an elongated ablation lesion comprising:
an elongated transmission line having a proximal portion suitable for connection to an energy source;
an antenna assembly coupled to the transmission line, and adapted to transmit energy therefrom sufficiently strong to cause tissue ablation,
a manipulating device cooperating with the antenna assembly for manipulative movement thereof;
a needle member having an elongated shaft and a distal tip portion adapted to pierce the tissue first surface and advance into the targeted biological tissue; and
a plurality of needle electrodes spaced apart along the elongated shaft, each said electrode being adapted to selectively transmit or receive electrical signals to measure at least one of conduction time, conduction velocity, phase angle, and impedance through at least a portion of the targeted biological tissue to determine the transmurality of an ablation lesion created therein.

31. The tissue ablation assembly according to claim 30, wherein
said needle electrodes are evenly spaced apart along the shaft.

32. The tissue ablation assembly according to claim 30, wherein
the spacings between adjacent needle electrodes are less at a distal portion of the shaft than between adjacent needle electrodes at a proximal portion of the shaft.

33. The tissue ablation assembly according to claim 30, wherein
said needle electrodes are ring electrodes.

34. The tissue ablation assembly according to claim 30, further including:
a first side electrode adapted to engage the tissue first surface at a location radially spaced from a longitudinal axis of the shaft to measure at least one of conduction time, conduction velocity, phase angle, and impedance with respect to one or more needle electrodes.

35. The tissue ablation assembly according to claim 34, wherein
said first side electrode is sufficiently radially spaced from the longitudinal axis of the shaft for positioning on one side of the elongated ablation lesion.

36. The tissue ablation assembly according to claim 34, further including:
a first support member extending radially away from the longitudinal axis of said shaft, and adapted to support said first side electrode such that when said shaft of the needle member is extended into the ablation lesion to position each needle electrode at the different respective depths of the biological tissue, said first side electrode engages the tissue first surface.

37. The device according to claim 36, wherein
said first support member is coupled to the shaft proximate a proximal portion thereof.

38. The tissue ablation assembly according to claim 36, wherein
said first support member extends substantially perpendicularly away from the longitudinal axis of said shaft.

39. The tissue ablation assembly according to claim 36, further including:
a second side electrode adapted to engage the tissue first surface at a location radially spaced from a longitudinal axis of the shaft, and spaced-apart from the first side electrode, to measure the at least one of conduction time, conduction velocity, phase angle, and impedance with respect to one or more needle electrodes.

40. The tissue ablation assembly according to claim 39, further including:
a first support member and a second support member each extending away from the longitudinal axis of said shaft, said first support member being adapted to support said first side electrode and said second support member being adapted to support said second side electrode such that when said shaft of the needle member is extended into the ablation lesion to position each needle electrode at the different respective depths of the biological tissue, at least one of said first side electrode and said second side electrode engages the tissue first surface.

41. The tissue ablation assembly according to claim 40 wherein
each proximal portion said first support member and said second support member are mounted to shaft about 180° apart along the longitudinal axis of the shaft.

42. The device according to claim 41, wherein
the proximal portions of the first support member and the second support member are coupled to the shaft proximate a proximal portion thereof.

43. The tissue ablation assembly according to claim 42, wherein
said first support member and said second support member each extends substantially perpendicularly away from the longitudinal axis of said shaft.

44. The tissue ablation assembly according to claim 43, wherein
said first side electrode is mounted proximate a distal tip portion of the first support member, and
said second side electrode is mounted proximate a distal tip portion of the second support member.

45. The tissue ablation assembly according to claim 44, wherein
said first side electrode is sufficiently radially spaced from the longitudinal axis of the shaft for positioning on one side of the elongated ablation lesion, and
said second side electrode is sufficiently radially spaced from the longitudinal axis of the shaft for positioning on an opposite side of the elongated ablation lesion.

46. The tissue ablation assembly according to claim 45, wherein
said needle electrodes are evenly spaced apart along the shaft.

47. The tissue ablation assembly according to claim 45, wherein
the spacings between adjacent needle electrodes are less at a distal portion of the shaft than between adjacent needle electrodes at a proximal portion of the shaft.

48. The tissue ablation assembly according to claim 45, wherein
said needle electrodes are ring electrodes.

49. The tissue ablation assembly according to claim 40, wherein
said energy source is selected from any one of microwave energy, RF energy, laser energy or cryogenic energy.

50. The tissue ablation assembly according to claim 30, wherein
said energy source is an electromagnetic energy such that the antenna assembly generates an electromagnetic field sufficiently strong to cause tissue ablation of the biological tissue.

51. The tissue ablation assembly according to claim 50, wherein
said antenna assembly includes a central axis and an elongated ablation region extending longitudinally along an exterior surface portion of the antenna assembly, said ablation region being adapted to be positioned substantially adjacent to or in engagement with the targeted biological tissue during operable use of the antenna assembly.

52. The tissue ablation assembly according to claim 51, wherein
said antenna assembly is adapted to direct a majority of the electromagnetic field generally in a predetermined direction across the ablation region.

53. The tissue ablation assembly according to claim 52, wherein
said antenna assembly includes an elongated antenna having a central axis off-set from the central axis of the antenna assembly.

54. The tissue ablation assembly according to claim 53, wherein
said antenna is off-set closer to the ablation region.

55. The tissue ablation assembly according to claim 52, wherein
said antenna assembly includes an elongated an antenna radially generating the electromagnetic field therefrom, and a shield device extending along the antenna to substantially shield a surrounding area of the antenna from the electromagnetic field radially generated therefrom while permitting a majority of the field to be directed generally in the predetermined direction.

56. A method for forming an elongated transmural lesion from a first surface of a targeted biological tissue to an opposed second surface thereof comprising:
manipulating an antenna assembly of an ablation instrument into engagement with or substantially adjacent to the tissue first surface;
generating an electromagnetic field from the antenna assembly sufficiently strong to cause tissue ablation;
before, during or after the generating, piercing a needle member having an elongated shaft into the targeted biological tissue from the tissue first surface, said needle member including a plurality of needle electrodes spaced apart along the elongated shaft; and
selectively transmitting or receiving electrical signals to measure from at least two needle electrodes at least one of conduction time, conduction velocity, phase angle, and impedance through at least a portion of the targeted biological tissue to determine the transmurality of an ablation lesion created or being created therein.

57. The method of claim 56, further including:
engaging a first side electrode with the tissue first surface at a location radially spaced from a longitudinal axis of the shaft; and
measuring at least one of conduction time, conduction velocity, phase angle, and impedance with respect to one or more needle electrodes.

58. The method of claim 57, further including:
analyzing the measured data of the at least one of conduction time, conduction velocity, phase angle, and impedance with respect to the one or more needle electrodes to determine the degree of tissue ablation.

59. The method of claim 57, further including:
engaging a second side electrode with the tissue first surface at a location radially spaced from a longitudinal axis of the shaft, and spaced-apart from the first side electrode, and
measuring at least one of conduction time, conduction velocity, phase angle, and impedance with respect to one or more needle electrodes.

60. The method of claim 59, wherein
said engaging a first side electrode is performed on one side of the elongated ablation lesion, and
said engaging a second side electrode is performed on an opposite side of the elongated ablation lesion.

61. The method of claim 60, further including:
analyzing the measured data of at least one of conduction time, conduction velocity, phase angle, and impedance with respect to the one or more needle electrodes to determine the degree of tissue ablation.

62. The method of claim 61, wherein
said needle member includes a first support member and a second support member each extending radially away from the longitudinal axis of said shaft, said first support member being adapted to support said first side electrode and said second support member being adapted to support said second side electrode, and
said piercing includes extending the shaft into the ablation lesion until the first side electrode and the second side electrode engages the first tissue surface.

63. A method for treating medically refractory atrial fibrillation of the heart comprising:
manipulating an antenna assembly of an ablation instrument into engagement with or substantially adjacent to a first surface of targeted cardiac tissue of the heart,
generating an electromagnetic field from the antenna assembly sufficiently strong to cause tissue ablation to form an elongated ablation lesion extending from the first surface toward an opposed second surface of the heart;
before, during or after the generating, piercing a needle member having an elongated shaft into the targeted cardiac tissue from the heart first surface, said needle member including a plurality of needle electrodes spaced apart along the elongated shaft;
selectively transmitting or receiving electrical signals from at least one needle electrode to measure at least one of conduction time, conduction velocity, phase angle, and impedance through at least a portion of the targeted cardiac tissue to determine the transmurality of the ablation lesion created or being created therein; and
repeating the manipulating, generating, piercing and transmitting or receiving to form a plurality of strategically positioned ablation lesions and/or to divide the left and/or right atria to substantially prevent reentry circuits.

64. The method of claim 63, further including:
engaging a first side electrode with the tissue first surface at a location radially spaced from a longitudinal axis of the shaft; and
measuring at least one of conduction time, conduction velocity, phase angle, and impedance with respect to one or more needle electrodes.

65. The method of claim 64, further including:
analyzing the measured data of the at least one of conduction time, conduction velocity, phase angle, and impedance with respect to the one or more needle electrodes to determine the degree of tissue ablation.

66. The method of claim 64, further including:
engaging a second side electrode with the tissue first surface at a location radially spaced from a longitudinal axis of the shaft, and spaced-apart from the first side electrode, and
measuring at least one of conduction time, conduction velocity, phase angle, and impedance with respect to one or more needle electrodes.

67. The method of claim 66, wherein
said engaging a first side electrode is performed on one side of the elongated ablation lesion, and
said engaging a second side electrode is performed on an opposite side of the elongated ablation lesion.

68. The method of claim 67, further including:
analyzing the measured data of at least one of conduction time, conduction velocity, phase angle, and impedance with respect to one or more needle electrodes to determine the degree of tissue ablation.

69. The method of claim 68, wherein
said needle member includes a first support member and a second support member each extending radially away from the longitudinal axis of said shaft, said first support member being adapted to support said first side electrode and said second support member being adapted to support said second side electrode, and
said piercing includes extending the shaft into the ablation lesion until the first side electrode and the second side electrode engages the first tissue surface.

70. The method of claim 63, wherein
the ablation lesions are strategically formed to create a predetermined conduction pathway between a sinoatrial node and an atrioventricular node of the heart.

71. The method of claim 63, wherein
said repeating the manipulating, generating, piercing and transmitting or receiving are applied in a manner isolating the pulmonary veins from the epicardium of the heart.

72. The method of claim 63, wherein
the heart remains beating throughout the manipulating, generating, piercing and transmitting or receiving.

73. The method of claim 63, wherein
said cardiac tissue includes the epicardium of the heart during a minimally invasive heart procedure.

74. The method of claim 63, further including:
arresting the patient's heart.
